(19) United States
(12) Patent Application Publication
(10) Pub. No.: US 2024/0050541 A1
(43) Pub. Date: Feb. 15, 2024

(54) INTRA-RENAL INFUSIONS OF C1 INHIBITOR PRIOR TO TRANSPLANTATION OF ALLOGRAFTS TO REDUCE DELAYED GRAFT FUNCTION AND ISCHEMIA/REPERFUSION INJURY AND TO IMPROVE LONG-TERM GRAFT FUNCTION

(71) Applicant: CEDARS-SINAI MEDICAL CENTER, Los Angeles, CA (US)

(72) Inventors: Stanley C. Jordan, Manhattan Beach, CA (US); Noriko Ammerman, Los Angeles, CA (US); Ashley Vo, Porter Ranch, CA (US); Irene Kim, Los Angeles, CA (US); Sanjeev Kumar, Los Angeles, CA (US)

(73) Assignee: CEDARS-SINAI MEDICAL CENTER, Los Angeles, CA (US)

(21) Appl. No.: 18/267,408

(22) PCT Filed: Dec. 21, 2021
(86) PCT No.: PCT/US21/64678

§ 371 Date: Jun. 14, 2023

Related U.S. Application Data
(60) Provisional application No. 63/128,757, filed on Dec. 21, 2020.

(51) Int. Cl. A61K 38/57; A61K 31/5377; A61K 31/522; A61K 31/43; A61K 31/573; A61P 37/06

(52) U.S. Cl. CPC A61K 38/57; A61K 31/5377; A61K 31/522; A61K 31/43; A61K 31/573; A61P 37/06

(57) ABSTRACT

The present invention describes method for reducing Delayed Graft Function (DGF) and Ischemia/Reperfusion Injury (IRI) by intra-renal infusion of a C1 esterase inhibitor.

### CROSS-REFERENCE TO RELATED APPLICATIONS

This application includes a claim of priority under 35 U.S.C. § 119(e) to U.S. provisional patent application No. 63/128,757, filed Dec. 21, 2020, the entirety of which is hereby incorporated by reference.

### FIELD OF INVENTION

This invention relates to the treatment of allografts and ischemia-reperfusion injury.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Delayed graft function (DGF), defined as the need for dialysis in the first week after kidney transplant, is estimated to occur in over 40% of deceased donor kidney transplants. Its development is associated with an increased risk of rejection, poorer long-term kidney allograft function, and lower patient and graft survival. This association is modified by the severity of DGF, as indicated by the duration of dialysis-dependence after transplant, where longer periods of dialysis-dependence are associated with progressively higher hazards of rejection and graft failure. Generally dialysis is needed at end stage kidney disease or kidney failure, often by the time a kidney loses about 85 to 90 percent of its function, or resulting in a subject having a glomerular filtration rate (GFR) of 15 (mL/min/1.73 m²) or less. Patients at stage 5 of kidney disease have a GFR of 15 mL/min or less and will need dialysis or a kidney transplant to live; and patients at stage 4 have a GFR between 15-29 mL/min and will likely need dialysis or a kidney transplant in the future. A recent study in Transplantation, 2021, 105(8):1808-1817 revealed that highly sensitized patients who experienced DGF after receiving positive-crossmatch kidney transplantation had a low amount of mean eGFR below 15 mL/min/1.73 m² at least from 24 hours to 21 days post-transplantation, and some able to discontinue dialysis with a duration of DGF lasting a range of 1-40 days post-transplantation; whereas highly sensitized patients from a same cohort who did not experience DGF after the transplantation, likely in association with the imlifidase treatment, had a functioning graft with a median eGFR of 47 mL/min/1.73 m² by end of the study at 180 days post-transplantation, whose eGFR quickly increased from below 15 mL/min/1.73 m² at 24 hours post-transplantation to about 45 mL/min/1.73 m² at Day 7 post-transplantation. Not surprisingly, kidneys at higher risk for DGF are more likely to be discarded in the United States despite the well-documented shortage in donor organ supply.

Accordingly, there is a need in the art for a treatment to improve allograft function and reduced DGF, and rejection and graft failure.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts an exemplary study of the present invention.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, N Y 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 10% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 9%, 8%, 7%, 6%, 5%, 4%, 3%, 2%, or 1% of that referenced numeric indication, if specifically provided for in the claims.

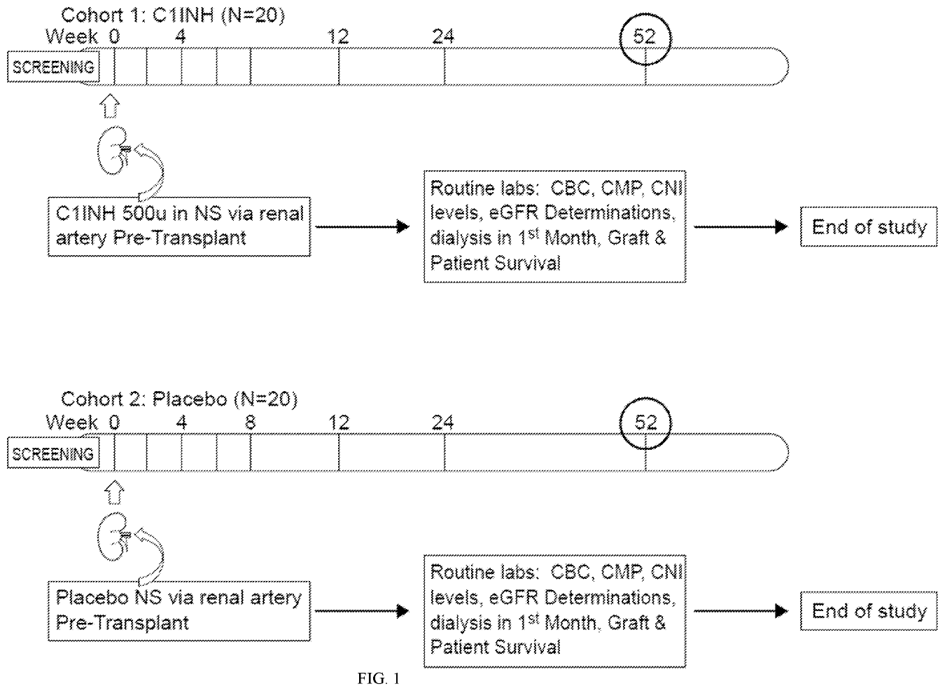

C1-esterase inhibitor (C1-INH) is a glycoprotein belonging to the superfamily of serine protease inhibitors called serpins. Human C1 esterase inhibitor is a protein of 500 amino acids, including a 22 amino acid signal sequence. An amino acid sequence of human C1 esterase inhibitor is provided in GenBank Accession No. CAA30314 (see also GeneID: 710, which also provides nucleotide sequences of the C1 esterase inhibitor); and also in FIG. 1 of U.S. Pat. No. 9,616,111, which is incorporated by reference herein in its entirety, wherein residues 1-22 are the signal sequence. In plasma, the C1 esterase inhibitor is a heavily glycosylated glycoprotein of approximately 76 kDa. The terms “C1 esterase Inhibitor,” “C1-Inhibitor,” “complement component 1 esterase inhibitor,” “complement factor 1 esterase inhibitor,” “C1INH,” and “C1-INH” are used interchangeably unless otherwise specified, and refer to the proteins or fragments thereof that function as serine protease inhibitors to inhibit proteases associated with the complement system, preferably proteases Clr and Cls as well as MASP-1 and MASP-2, with the kallikrein-kinin system, preferably plasma kallikrein and factor XIIa, and with the coagulation system, preferably factor XIa. In addition, C1-INH can serve as an anti-inflammatory molecule that reduces the selectins-mediated leukocyte adhesion to endothelial cells. C1-INH as used here can be a native serine protease inhibitor or active fragment thereof, or it can comprise a recombinant peptide, a synthetic peptide, peptide mimetic, or peptide fragment that provides similar functional properties—e.g., the inhibition of proteases Clr and C1 s, and/or MASP-1 and MASP-2 and/or factor XIIa and/or factor XIa. Further disclosure regarding the structure and function of C1-Inhibitor is seen in U.S. Pat. Nos. 4,915,945; 5,939,389; 6,248,365; 7,053,176; and WO 2007/073186, which are hereby incorporated by reference in their entirety.

C1 esterase inhibitor can be produced according to methods known to one of skill in the art. For example, plasma-derived C1-INH can be prepared by collecting blood plasma from several donors. Donors of plasma should be healthy as defined in the art. Preferably, the plasma of several (1000 or more) healthy donors is pooled and optionally further processed. An exemplary process for preparing C1-inhibitor for therapeutic purposes is disclosed in U.S. Pat. No. 4,915,945, the disclosure of which is hereby incorporated by reference in its entirety. Alternatively, in some embodiments C1-INH can be collected and concentrated from natural tissue sources using techniques known in the art. Commercially available products comprising C1INH are, e.g. plasma-derived CINRYZE® (Viropharma), recombinant RUCONEST® or RHUCIN® (both Pharming), and plasma-derived BERINERT® (CSL Behring). Recombinant C1-INH can be prepared by known methods.

Historically DGF has been defined as the requirement for dialysis during the first week after renal transplantation, however the postoperative requirement of hemodialysis or peritoneal dialysis is not standardized and the decision to dialyze varies from center to center and among consultants. Efforts have been made to scientifically quantify DGF in a more stringent manner with various alternative definitions of DGF including. the number of days to achieve a creatinine clearance of >10 mL/min, calculated by the Gault-Cockroft formula, a serum creatinine level of >3 mg/dL on the fifth day post-transplant, the need for dialysis within 72 hours after transplantation serum creatinine level changes, including an increase, remaining unchanged, or decreasing by less than 10% per day immediately after surgery during three consecutive days for >1 week, a rising serum creatinine level above that before surgery, or urine output of <300 mL within 6 hours of transplantation, despite diuretics and adequate volume urine output of <1 L in the first 24 hours, or a decrease in serum creatinine of <20-30% reflected in a poor glomerular filtration rate (GFR), and calculating the creatinine reduction ratio on day two following surgery. However, despite these alternative suggestions, the conventional definition of “requirement for dialysis in the first week post-transplant” remains the most utilized and published definition for DGF, with “need for dialysis” being easily measured and clinically relevant.

The Kidney Donor Profile Index (KDPI) is a numerical measure that combines ten donor factors, including clinical parameters and demographics, to summarize into a single number the quality of deceased donor kidneys relative to other recovered kidneys. Generally, the KDPI is a measure of how long a deceased donor kidney is expected to function relative to all of the kidneys recovered in the U.S. during the last year. Lower KDPI scores are associated with longer estimated function, while higher KDPI scores are associated with shorter estimated function. For example, a kidney with a KDPI of 20% is expected to have shorter longevity than 20% of recovered kidneys (i.e., longer function than 80% of recovered kidneys). The KDPI is derived by first calculating the Kidney Donor Risk Index (KDRI) for a deceased donor.

Kidneys from a donor with a KDPI of 90%, for example, have a KDRI (which indicates relative risk of graft failure) greater than 90% of recovered kidneys. The KDPI is a mapping of the KDRI from a relative risk scale to a cumulative percentage scale. The reference population used for this mapping is all deceased donors in the United States with a kidney recovered for the purpose of transplantation in the prior calendar year. Lower KDPI values are associated with increased donor quality and expected longevity.

Early graft function has a long-term effect on graft survival. Poor early graft function and DGF contributes to decreased short- and long-term patient and graft survival, increased incidence of acute rejection, prolonged hospitalization, and higher costs of transplantation. Although multiple factors contribute to the impaired graft function, ischemia-reperfusion injury (IRI) is the underlying pathophysiology leading to poor early graft function and DGF. A >35% incidence of DGF has remained constant over time despite significant improvements in immunosuppressive strategies and patient management. This may be due to increased use of kidneys from “extended-criteria” and/or non-heart-beating donors, where even greater rates (>60%) of DGF have been reported.

More than 95,037 people in 2019 are waiting for a kidney transplant in the United States. Of the 19,360 kidney transplants performed in the US in 2018, 20% were from DCD donors and 9% from donors of KDPI>85%. The USRDS reports that more than 50% of patients on the waiting list are willing to accept a kidney from an expanded-criteria donor (KDPI>85%). Described herein embodiments of the invention expand the use of high KDPI kidneys and reduce wastage by showing improved function after C1INH treatment.

Described in further detail herein, there is a study group of about 40 patients. Treatment Arm I will have kidneys from donors with a KDPI>80%, each kidney of which will be infused with one intrarenal dose of 500 U of BERINERT® (a C1 esterase inhibitor, C1INH) in or prior to implantation into the recipient. Recipients will receive C1NH 500 U/kidney infused into the renal artery pre-operatively or intra-operatively at any one point in time, from time of induction of anesthesia up to the beginning of renal vein anastomosis before graft reperfusion. Control Arm will have kidneys from donors with a KDPI>80%, each kidney of which will be administered one intrarenal dose of normal saline (NS; e.g., 0.9% normal saline) in a volume identical to the volume of the dose of BERINERT® before implantation of kidney into the patient. Drug v. placebo administration are randomized 1:1.

Accordingly, various embodiments of the present invention provide for method of reducing delayed graft function, ischemia/reperfusion injury, or improving long-term graft function.

Various embodiments of the present invention provide for a method of reducing delayed graft function, comprising: administering a therapeutically effective dose of complement component 1 esterase inhibitor (C1INH) to an allograft prior to implantation of the allograft into a subject in need thereof. Further embodiments of the present invention provide for a method of reducing delayed graft function, comprising: administering a therapeutically effective dose of complement factor 1 esterase inhibitor (C1INH) to an allograft before graft reperfusion in the recipient, such as from the time of induction of anesthesia to the beginning of renal vein anastomosis, or before clamp removal for reperfusion of a kidney allograft in the recipient. In various embodiments, the methods of reducing delayed graft function result in, or are alternatively expressed as for, reducing a need for dialysis in the first post-transplantation week; or increasing GFR or eGFR by about 5, 10, 15, 20, 25, or 30 mL/min/1.73 m² or more by 7 days post-transplantation, compared to a baseline level prior to transplantation or within 24 hours of transplantation. In some aspects, the methods result in, or are alternatively expressed as for, increasing GFR or eGFR in the recipient by about 5, 10, 15, 20, 25, or 30 mL/min/1.73 m² or more, or obtaining a functioning allograft in the recipient, at 6 months, 9 months, or 1 year post-transplantation. In other aspects, the methods of reducing delayed graft function result in reduction of a need for dialysis in the first 30 days post-transplantation. In various embodiments, the methods of reducing delayed graft function relates to, or are alternatively described as, reducing the severity or likelihood of ischemia reperfusion injury (IRI). IRI involves activation of cell death programs (necrosis, apoptosis, autophagy), endothelial dysfunction (swelling of ECs, degradation of cytoskeleton, increasing vascular permeability, causing vasoconstriction and potentially no reflow despite reperfusion), and transcriptional reprogramming. Ischemic injury may arise to the allograft when it is removed from a donor and before it is reperfused in a recipient. During reperfusion, oxygen levels increase, and the pH normalizes which is harmful for the previously ischemic cells. In various aspects, IRI is different from immune responses such as antibody-mediated rejection of transplant. In various implementations, the methods disclosed herein reduces severity, duration, or likelihood of IRI when allograft is transplanted, thereby reducing likelihood of non- or delayed graft function.

Various embodiments of the present invention provide for a method of reducing ischemia/reperfusion injury, comprising: administering a therapeutically effective dose of complement component 1 esterase inhibitor (C1INH) to an allograft prior to implantation of the allograft into a subject in need thereof. Further embodiments of the present invention provide for a method of reducing ischemia/reperfusion injury, comprising: administering a therapeutically effective dose of complement component 1 esterase inhibitor (C1INH) to an allograft before graft reperfusion in the recipient, such as from the time of induction of anesthesia to the beginning of renal vein anastomosis, or before clamp removal for reperfusion of a kidney allograft in the recipient.

Various embodiments of the present invention provide for a method of improving long-term graft function, improving early graft function, or improving graft survival, wherein the method comprises: administering a therapeutically effective dose of complement component 1 esterase inhibitor (C1INH) to an allograft prior to implantation of the allograft into a subject in need thereof. Further embodiments of the present invention provide for a method of improving long-term graft function, improving early graft function, or improving graft survival, comprising: administering a therapeutically effective dose of complement component 1 esterase inhibitor (C1INH) to an allograft after graft implantation (e.g., renal aorta and/or vein anastomosis) but before graft reperfusion in the recipient, such as from the time of induction of anesthesia to the beginning of renal vein anastomosis, or before clamp removal for reperfusion of a kidney allograft in the recipient. In some aspects, early graft condition (function or survival) includes the first one, two, or three months post-transplantation; and in some aspects long term graft condition (function or survival) refers to one, two, or three years, or longer, after transplantation. In various aspects, the methods for improving graft function, wherein graft is a kidney, result in, or can be alternatively termed as, inducing initial postoperative decline in serum creatinine, improving GFR or estimated GFR (e.g., at 12 month post-transplant) or creatinine clearance, reducing a need for dialysis (e.g., in first post-operative week, or in first 30 days post-operation), reducing rejection episodes (e.g., reduction by 10%, 20%, 30%, or more in the number of rejection episodes in first year post operation), and/or reducing level of donor specific antibodies (DSA).

In various implementations, the improvement and/or the reduction is compared to respective parameters in a recipient of an allograft that has not been infused with a C1INH prior to implantation or prior to reperfusion of the allograft in the recipient. In further implementations, the improvement and/or the reduction results in a level of function and/or survival of the allograft with KDPI>80% or allograft from an extended criteria donor in a recipient, wherein the level of function and/or survival is comparable to that of an allograft from a living donor or one with a low KDPI score (e.g., KDPI of 0-20%). In yet another implementation, the improvement and/or the reduction is compared to a reference value calculated based on a plurality of recipients, each receiving an allograft that has not been infused with a C1INH prior to implantation or prior to reperfusion of the allograft in the recipient, and the plurality of recipients are 2-25, 10-30, 10-50, 20-50, 50-100, 100-200, 200-300, 300-400, 400-500, 500-750, 750-1000, or at least 1000 recipients in number.

In various embodiments, administering the therapeutically effective dose of C1INH to the allograft (e.g., kidney) comprises infusing into the renal artery.

In various embodiments, the C1INH is BERINERT® complement component 1 inhibitor. BERINERT is sourced from human plasma. In various embodiments, the C1INH is plasma-derived BERINERT® (CSL Behring), plasma-derived CINRYZE® (Viropharma), or recombinant RUCONEST® or RHUCIN® (both Pharming; brand name is RUCONEST®, previous name RHUCIN®, generic name: recombinant C1 esterase inhibitor).

BERINERT® is a human plasma-derived, purified, pasteurized, lyophilized concentrate of C1 esterase inhibitor. BERINERT® is prepared from large pools of human plasma from US donors. Generally only plasma that has passed virus screening is used in production of BERINERT®, and the limit for Parvovirus B19 in the fractionation pool is set not to exceed 104 IU of Parvovirus B19 DNA per mL. The production of BERINERT® includes virus inactivation/reduction of three steps: Pasteurization in aqueous solution at 60° C. for 10 hours; Hydrophobic interaction chromatography; Virus filtration (also called nanofiltration) by two filters, 20 nm and 15 nm, in series.

RUCONEST® is a recombinant analogue of human complement C1 esterase inhibitor. RUCONEST® is purified from the milk of transgenic rabbits.

CINRYZE® is a sterile, stable, lyophilized preparation of C1 esterase inhibitor derived from human plasma. CINRYZE is manufactured from human plasma purified by a combination of filtration and chromatographic procedures. The specific activity of CINRYZE is 4.0 to 9.0 U/mg protein. The purity is ≥90% human C1 esterase inhibitor.

Feussner et al. in Transfusion 2014; 54:2566-2573 is incorporated by reference herein in its entirety, which summarized the difference in manufacturing and further studied the purity and function of BERINERT, CINRYZE, and RUCONEST. Specifically, BERINERT is evaluated to contain fewer high-molecular-weight proteins than CINRYZE. In various aspects, BERINERT contains at least 94% purity of C1INH, wherein non-C1INH proteins in BERINERT is about 3.2% or less than 6% (e.g., the high-molecular-weight proteins is less than about 3% and low-molecular weight proteins is about 0.2%); whereas CINRYZE contains about 65-75% purity of C1INH when evaluated with the standard BERINERT method at an elution time of 13.5 to 15.0 minutes, or about 87-92% purity of C1INH when evaluated with the adapted BERINERT method to cover peak split in CETOR and CINRYZE at an elution time of 13.5 to 17.0 minutes; wherein non-C1INH proteins in CINRYZE is about 10% or at least 10% (e.g., the high-molecular-weight proteins in CINRYZE is about 10%, and the low-molecular-weight proteins about 0.2%).

In some embodiments, the C1INH is BERINERT®; and the C1INH is not CINRYZE® or RUCONEST®, or RHUCIN®. In some embodiments, the pharmaceutical composition for use in the disclosed methods is reconstituted from powder having at least 94% C1INH protein. In some embodiments, the pharmaceutical composition for use in the disclosed methods is reconstituted from powder having at least 95% C1INH protein. In some embodiments, the pharmaceutical composition for use in the disclosed methods is reconstituted from powder having at least 96% C1INH protein. In some embodiments, the pharmaceutical composition for use in the disclosed methods is reconstituted from powder having at least 97% C1INH protein. In some embodiments, the pharmaceutical composition for use in the disclosed methods is reconstituted from powder having at least 98% or 99% C1INH protein. In some embodiments, the pharmaceutical composition for use in the disclosed methods is reconstituted from powder having about 90%, 91%, 92%, 93% of C1INH protein or higher.

In some embodiments, the C1-INH composition for use in the disclosed methods in an un-reconstituted form has a concentration of at least 90%, 91%, 92%, 93%, 94%, 95%, 96%, 97%, 98%, or 99% C1-INH. In some embodiments, the C1-INH composition for use in the disclosed methods is derived or isolated from human plasma. In an un-reconstituted form, non-C1INH protein is less than 9%, 8%, 7%, 6%, 5%, 4%, 3%, 2% or 1% in the C1-INH composition derived from human plasma.

In some embodiments, the pharmaceutical composition for use in the disclosed methods contains C1INH protein and non-C1INH, plasma derived proteins at a ratio of at least 94:6, that is 16:1 or greater. In some, the pharmaceutical composition for use in the disclosed methods contains C1INH protein and non-C1INH proteins at a ratio of between 16:1 and 100:1. In some, the pharmaceutical composition for use in the disclosed methods contains C1INH protein and non-C1INH proteins at a ratio of at least 20:1, such as between 20:1 and 100:1. In some, the pharmaceutical composition for use in the disclosed methods contains C1INH protein and non-C1INH proteins at a ratio of at least 24:1, such as between 24:1 and 100:1. In various aspects, the percentage, or ratio of two percentages resulting therefrom, is based on the fraction of total AUC, area under a chromatography curve, in separating molecules in a composition. In other aspects, the percentage is based on weight fraction in a composition.

In some embodiments, the C1INH is a human C1 esterase inhibitor. In further embodiments, the C1INH is a plasma-derived C1 inhibitor. In other embodiments, the C1INH is a recombinant C1 inhibitor. In additional embodiments, the C1INH is identical with the naturally occurring human protein or a variant thereof. The C1-INH shall encompass all natural occurring alleles which have the same function as the C1-inhibitor. A C1INH for use in the methods of the instant invention may have an amino acid sequence that has at least 65, 70, 75, 80, 85, 90, 95, 98, 99, or 100% identity with the amino acid sequence of human C1 esterase inhibitor. In some embodiments, the C1INH comprises an amino acid sequence at least 95% identical to human C1 esterase inhibitor. In some embodiments, the C1INH comprises an amino acid sequence at least 95% identical to human C1 esterase inhibitor that excludes the signal sequence.

The C1 esterase inhibitor may be isolated or purified from plasma (e.g., human plasma) or recombinantly produced. When purified from plasma, the C1 esterase inhibitor may be nanofiltered and pasteurized.

Additional embodiments provide the C1-inhibitor herein is modified to improve bioavailability and/or half-life, to improve efficacy, and/or to reduce potential side effects. The modification can be realized by recombinant or other steps. Examples for such a modification can be a glycosylation or an albumin fusion of the described C1-inhibitor. Further disclosure regarding the glycosylation and the albumin fusion of proteins is seen in US20030171267, which is hereby incorporated by reference in their entirety.

In various embodiments, the therapeutically effective dose of C1INH is about 100-1000 U/allograft. In various embodiments, a dose of C1INH about 500-1000 U/kidney is infused to the renal artery prior to transplantation surgery. One standard unit of C1 esterase inhibitor concentrate is equal to the amount of C1 esterase inhibitor in 1 mL of fresh citrated human plasma, which is equivalent to 270 mg/L or 2.5 M/L. In various embodiments, the therapeutically effective dose of C1INH is about 100, 150, 200, 250, 300, 350, 400, 450, 500, 550, 600, 650, 700, 750, 800, 850, 900, 950 or 1000 U/allograft. In various embodiments, the therapeutically effective dose of C1INH is about 250 U/allograft. In various embodiments, the therapeutically effective dose of C1INH is about 300 U/allograft. In various embodiments, the therapeutically effective dose of C1INH is about 400 U/allograft. In various embodiments, the therapeutically effective dose of C1INH is about 500 U/allograft. In various embodiments, the therapeutically effective dose of C1INH is about 600 U/allograft. In various embodiments, the therapeutically effective dose of C1INH is about 700 U/allograft. In various embodiments, the therapeutically effective dose of C1INH is about 750 U/allograft. In additional embodiments, the therapeutically effective dose of C1INH is about 50-100 U/allograft. In additional embodiments, the therapeutically effective dose of C1INH is about 100-150 U/allograft. In additional embodiments, the therapeutically effective dose of C1INH is about 150-200 U/allograft. In additional embodiments, the therapeutically effective dose of C1INH is about 200-250 U/allograft. In additional embodiments, the therapeutically effective dose of C1INH is about 250-300 U/allograft. In additional embodiments, the therapeutically effective dose of C1INH is about 300-400 U/allograft. In additional embodiments, the therapeutically effective dose of C1INH is about 400-500 U/allograft. In additional embodiments, the therapeutically effective dose of C1INH is about 500-600 U/allograft. In additional embodiments, the therapeutically effective dose of C1INH is about 350-550 U/allograft. In additional embodiments, the therapeutically effective dose of C1INH is about 450-650 U/allograft. In additional embodiments, the therapeutically effective dose of C1INH is about 60-700 U/allograft. In additional embodiments, the therapeutically effective dose of C1INH is about 700-800 U/allograft. In additional embodiments, the therapeutically effective dose of C1INH is about 800-900 U/allograft. In additional embodiments, the therapeutically effective dose of C1INH is about 900-1000 U/allograft. In various embodiments, the therapeutically effective dose of C1INH is up to 1000 U/kidney allograft prior to transplantation or prior to reperfusion in the recipient.

In various embodiments, the therapeutically effective dose of C1INH is administered to the allograft about 1-2 hours prior to implantation of the allograft. In some embodiments, the C1INH is infused into the allograft over a period of time of about 5 minutes, 10 minutes, 20 minutes, 30 minutes, 40 minutes, 50 minutes, 1 hour, 1.5 hours, or 2 hours. In some embodiments, the allograft before reperfusion has been infused with the C1INH for about 5 minutes, 10 minutes, 20 minutes, 30 minutes, 40 minutes, 50 minutes, 1 hour, 1.5 hours, or 2 hours prior to implantation or prior to reperfusion in the recipient. In some embodiments, the allograft infused with the C1INH is reperfused immediately or within 5 minutes, 10 minutes, 20 minutes, 30 minutes, 40 minutes, 50 minutes, 1 hour, 1.5 hours, or 2 hours from the completion of the C1INH infusion. In some embodiments, BERINERT® in a single-use vial that contains 500 units of C1 esterase inhibitor as a lyophilized concentrate is reconstituted with 10 mL of diluent (e.g., sterile water), and after reconstitution, administration is performed within 8 hours provided the solution is stored at up to 25° C. without freezing or refrigeration, and the administration is performed 60-120 minutes before graft reperfusion. In some embodiments, the C1INH is reconstituted or present in a composition in a concentration of about 50 U/mL, or at least 50 U/mL, at least 40 U/mL, at least 25 U/mL, or at least 10 U/mL. Preferably, C1INH is reconstituted immediately or shortly before administration, as the half-life of BERINERT® is about 18.4 to 21.9 hours and the half-life of CINRYZE® is about 56±36 hours. In various embodiments, these methods further comprise administering saline to the allograft prior to administering the therapeutically effective dose of C1INH to the allograft. In various embodiments, the saline is heparinized normal saline. In further embodiments, the heparinized normal saline is a sterile salt water solution containing 10-100 U heparin/mL and about 0.9% sodium chloride. In various embodiments, the saline is heparin saline, which may contain a concentration of 10-100 U heparin/mL. In various embodiments, the saline is normal saline, i.e., a sterile salt water solution containing 0.9% sodium chloride.

In various embodiments, these methods further comprise implanting the allograft into a subject in need thereof. In further embodiments, these methods further comprise performing reperfusion of the allograft in the subject following the administration of the C1INH to the allograft. Performing perfusion of the allograft in the subject after transplantation, called “reperfusion,” includes passing fluid through the circulatory system or lymphatic system to the transplanted organ, usually referring to the delivery of blood to a capillary bed in the organ. This is different from infusing or “perfusing” an allograft prior to transplantation—wherein the perfusion fluid is used to perfuse and preserve the kidney prior to transplantation and acts as a medium in which organisms can grow—and in various embodiments of the invention here, C1INH is added to the perfusion fluid for infusing the allograft prior to transplantation or prior to reperfusion of the allograft in a recipient. In various implementations, a C1INH solution is used to perfuse or infuse the allograft prior to transplantation or prior to blood perfusion of the allograft with the recipient's circulatory system, In some embodiments of these methods, the allograft is administered with a therapeutically effective amount of C1INH, such that C1INH in a sufficient dose and a sufficient time binds all or most or substantially all endothelial surfaces in the transplanted allograft, followed by performing reperfusion of the allograft in the recipient.

In various embodiments, these methods further comprise administering immunosuppression therapy to the subject.

In various embodiments, immunosuppression therapy comprises induction antibody therapy. In various embodiments, the induction antibody therapy is THYMOGLOBULIN® or Campath 1H. In various embodiments, the induction antibody therapy is THYMOGLOBULIN® and the first dose of THYMOGLOBULIN® is administered immediately after the transplantation of the allograft followed with additional doses of THYMOGLOBULIN®. In various embodiments, the total dose THYMOGLOBULIN® is about 6 mg/kg. In various embodiments, the induction antibody therapy is Campath 1H and is administered in a single 30 mg subcutaneous dose post-transplant. In further embodiments, the immunosuppression therapy does not include IL-2 receptor inhibitors (e.g., daclizumab, basiliximab).

In various embodiments, immunosuppression therapy comprises calcineurin inhibitor (e.g., tacrolimus, cyclosporine), mycophenolate mofetil (MMF), or corticosteroid. Exemplary corticosteroids include but are not limited to cortisone, prednisone, prednisolone, methylprednisolone, dexamethasone, betamethasone, and hydrocortisone.

In some embodiments, these methods further comprise administering C1INH to the subject before the subject received the transplant, e.g., about one day before transplantation, on the day of and before transplantation, or both. In other embodiments, these methods further comprise administering C1INH to the subject at the time of transplantation, at about one day post transplantation, or both, or following the transplantation. In yet additional embodiments, these methods further comprise administering C1INH to the subject before the subject received the transplant and one or both of at the time of transplantation and at least about one day post transplantation.

Administration to a patient may occur in a single dose or in repeated administrations, and in any of a variety of physiologically acceptable salt forms, and/or with an acceptable pharmaceutical carrier and/or additive as part of a pharmaceutical composition. In various embodiments, the C1INH is administered in one dose of a therapeutically effective amount to the allograft on day 0 of implantation which is before reperfusion of the allograft in the recipient. In various embodiments, the therapeutically effective amount of C1INH is administered to the allograft once on day 0 before reperfusion in the recipient and repeated for a second time about 24 hours later.

The dosage may be determined by a physician and adjusted, as necessary, to suit the observed effects of the treatment. In certain embodiments, the dose of C1-INH may range from approximately 1 U/kg to 5000 U/kg of bodyweight. In various embodiments, the dose of C1-INH for a subject is 1, 5, 7.5, 10, 15, 20, 25, 30, 50, 75, 100, 125, 150, 175, 200, 225, 250, 275, 300, 325, 350, 375, 400, 450, 500, 1000, 1500, 2000, 2500, 3000, 3500, 4000, or 4500 U/kg of bodyweight (or any value in between). In some embodiments, the dose of C1-INH for a subject is between 10 and 50, between 20 and 50, between 25 and 50, or between 30 and 75 U/kg of bodyweight. The potency of C1 esterase inhibitor is also expressed in International Units (IU). In some embodiments, the dose of BERINERT is one of 300, 400, 450, 500, 550, 600, 650, 700, 750, 800, 850, 900, 950, or 1000 IU/allograft, or any ranges in between the numbers; and when applicable, one dose of BERINERT is one of 20, 25, 30, 35, 40, 45, or 50 IU/kg of patient, administered intravenously, or administered in another route at a dose equivalent to the intravenous dose. In some embodiments, the dose of C1-INH for a subject is one of 10, 20, 30, 40, 50, 60, 70, 80, 90 or 100 U/kg. In some embodiments, the dose of C1-INH for a subject is one of 15, 25, 35, 45, 55, 65, 75, 85, or 95 U/kg. In some embodiments, the dose of C1-INH for a subject is one of 15, 20, 25, 30, 35, 40, 45, 50, 55, or 60 U/kg.

In various implementations, the C1INH is administered to a transplant recipient at about 20-50 U/kg of the recipient intravenously (i.v.), or via another route of administration and at an equivalent dose to i.v. 20-50 U/kg, prior to transplanting to the recipient the kidney allograft having been infused with about 10-1000 U C1INH/allograft. In various implementations, the C1INH is administered to a transplant recipient at about 20-50 U/kg of the recipient intravenously (i.v.), or via another route of administration and at an equivalent dose to i.v. 20-50 U/kg, at time of transplantation or following transplantation to the recipient of the kidney allograft that has been infused with about 10-1000 U C1INH/allograft. In further implementations, the C1INH is administered to a transplant recipient at about 20-50 U/kg of the recipient intravenously (i.v.), or via another route of administration and at an equivalent dose to i.v. 20-50 U/kg, both before transplantation and following transplantation to the recipient of the kidney allograft that has been infused with about 10-1000 U C1INH/allograft.

In some embodiments, these methods do not comprise administering C1INH to the subject before transplantation, at the time of transplantation, or following the transplantation. For example, the methods comprise infusing the allograft with C1INH as a monotherapy.

In various embodiments, these methods further comprise administering an anti-infective to the subject to reduce the likelihood of the subject having an infection. In various embodiments, the anti-infective is an anti-viral, an anti-fungal or an antibiotic. In various embodiments, the anti-infective is valgancyclovir, ganciclovir, or penicillin. Exemplary anti-viral drugs include but are not limited to abacavir, acyclovir, adefovir, amantadine, ampligen, amprenavir, umifenovir, atazanavir, atripla, baloxavir marboxil, biktarvy, boceprevir, bulevirtide, cidofovir, cobicistat, combivir, daclatasvir, darunavir, delavirdine, descovy, didanosine, docosanol, dolutegravir, doravirine, edoxudine, efavirenz, elvitegravir, emtricitabine, enfuvirtide, entecavir, etravirine, famciclovir, fomivirsen, fosamprenavir, foscarnet, ganciclovir, ibacitabine, ibalizumab, idoxuridine, imiquimod, imunovir, indinavir, lamivudine, letermovir, lopinavir, loviride, maraviroc, methisazone, moroxydine, nelfinavir, nevirapine, nexavir, nitazoxanide, norvir, oseltamivir, penciclovir, peramivir, penciclovir, peramivir, pleconaril, podophyllotoxin, raltegravir, remdesivir, ribavirin, rilpivirine, rimantadine, ritonavir, saquinavir, simeprevir, sofosbuvir, stavudine, taribavirin, telaprevir, telbivudine, tenofovir alafenamide, tenofovir disoproxil, tipranavir, trifluridine, trizivir, tromantadine, truvada, umifenovir, valaciclovir, valganciclovir, vicriviroc, vidarabine, zalcitabine, zanamivir, and zidovudine. Exemplary anti-fungal agents include but are not limited to clotrimazole, econazole, miconazole, terbinafine, fluconazole, ketoconazole, nystatin, and amphotericin. Exemplary antibiotics include but are not limited to penicillins (e.g., penicillin, amoxicillin), cephalosporins (e.g., cephalexin), macrolides (e.g., erythromycin, clarithromycin, azithromycin), fluoroquinolones (e.g., ciprofloxacin, levofloxacin, ofloxacin), sulfonamides (e.g., co-trimoxazole, trimethoprim), tetracyclines (e.g., tetracycline, doxycycline), and aminoglycosides (e.g., gentamicin, tobramycin).

In various embodiments, a method of reducing delayed graft function, reducing ischemia/reperfusion injury, and/or improving long-term graft function, consists essentially of administering a therapeutically effective dose of complement component) esterase inhibitor (C1INH) to an allograft before graft reperfusion in the recipient, and administering one or more immunosuppression therapy, one or more anti-infectives, or both.

In various embodiments, the methods of reducing delayed graft function, reducing ischemia/reperfusion injury, and/or improving long-term graft function, further comprises performing one or more of: complete blood count (CBC), comprehensive metabolic panel (CMP) test, assaying calcineurin inhibitor (CNI) level, assaying GFR or eGFR level, dialysis, and determining graft and/or patient survival.

In various embodiments, a method of reducing delayed graft function, reducing ischemia/reperfusion injury, and/or improving long-term graft function, consists essentially of administering a therapeutically effective dose of complement component 1 esterase inhibitor (C1INH) to an allograft before graft reperfusion in the recipient; administering one or more immunosuppression therapy, one or more anti-infectives, or both; and performing one or more of: complete blood count (CBC), comprehensive metabolic panel (CMP) test, assaying calcineurin inhibitor (CNI) level, assaying GFR or eGFR level, dialysis, and determining graft and/or patient survival.

“Allograft” refers to the transplant of an organ, tissue, or cells from one individual to another individual of the same species who is not an identical twin. In various embodiments, the methods disclosed herein for the transplant are not used for an autograft; i.e., not for transplantation of an organ or tissue or cells from and to the same individual.

In various embodiments, the allograft is a kidney. In various embodiments, the kidney a kidney allograft having a kidney donor profile index (KDPI) greater than 80%. In various embodiments, the KDPI is greater than 85%. In various embodiments, the KDPI is greater than 86%. In various embodiments, the KDPI is greater than 87%. In various embodiments, the KDPI is greater than 88%. In various embodiments, the KDPI is greater than 89%. In various embodiments, the KDPI is greater than 90%. In various embodiments, the allograft is from an extended-criteria donor, or from a non-heart-beating donor. In various embodiments, the allograft is from a deceased donor who is high risk for DGF, e.g., a deceased donor with a KDPI of greater than or at least 80%, 85%, or 90%.

In additional embodiments, the allograft is procured from a donor after cardiac death, a donor on hemodialysis or renal replacement therapy prior to death of the donor or procurement of the donor's organ, or an allograft with a cold ischemia time (CIT) longer than acceptable CIT limits (e.g., 4-6 hours for heart, <12 hours for liver and pancreas, <24 hours for kidney). In some embodiments, the allograft is a kidney from a donor after cardiac death. In some embodiments, the allograft is a kidney from a donor on hemodialysis or continuous renal replacement therapy (HD/CRRT). In some embodiments, the allograft is a kidney with a CIT of 24 hours or longer. Typically, graft cold ischemia time (CIT) is defined as the interval from initiation of donor in vivo cold organ preservation to removal of the graft from 4° C. cold storage. In some embodiments, the allograft is a kidney with a CIT of 30 hours or longer. In some embodiments, the allograft is a kidney with a CIT of 36 hours or longer. In some embodiments, the allograft is a kidney with a CIT of 42 hours or longer. In some embodiments, the allograft is a kidney with a CIT of 48 hours or longer. In some embodiments, the allograft is a kidney with a CIT of about 24, 25, 26, 27, 28, 29, 30, 31, 32, 33, 34, 35, 36, 37, 38, 39, 40, 41, 42, 43, 44, 45, 46, 47, 48, 72, 96 hours, or any length of time in between the specified hours. In some embodiments, the allograft is a kidney with donor terminal serum creatinine level of 3.0 mg/dL or greater. In some embodiments, the allograft is a kidney with donor terminal serum creatinine level of 3.5 mg/dL or greater. In some embodiments, the allograft is a kidney with donor terminal serum creatinine level of 4 mg/dL or greater. In some embodiments, the allograft is a kidney with donor terminal serum creatinine level of 4.5 mg/dL or greater. In some embodiments, the allograft is a kidney with donor terminal serum creatinine level of 5 mg/dL or greater. In some embodiments, the allograft is a kidney with donor terminal serum creatinine level of 6 mg/dL or greater. In some embodiments, the allograft is a kidney with donor terminal serum creatinine level of 8 mg/dL or greater. In some embodiments, the allograft is a kidney with donor terminal serum creatinine level of 10 mg/dL or greater.

In other embodiments, the allograft is an organ from a living donor. In further embodiments, the allograft is a kidney from a living donor.

In various embodiments, the allograft is a heart, liver, lung, small bowel, pancreas or bone marrow.

Various embodiments provide that the subject, patient, or recipient, as used interchangeably unless otherwise specified, is a human, that is, the allograft recipient is a human. Other embodiments provide that the subject is a mammalian animal. In various embodiments, the subject in the methods disclosed herein is in need of transplantation of an organ, e.g., in need of kidney transplantation, who in some embodiments have a GFR of 15 mL/min or less before transplantation.

In certain embodiments, a pharmaceutical composition comprising C1INH is prepared or provided for use in infusing an allograft prior to implantation or after implantation but prior to reperfusion in a recipient. For example, if a powder or lyophilized form of C1-INH (e.g., by freeze drying) is provided and an aqueous pharmaceutical is desired, the powder can be dissolved by mixing with aqueous components of the pharmaceutical formulation and stirred using suitable techniques such as vortexing or gentle agitation. In other embodiments, C1-INH is provided in lyophilized form and combined with aqueous pharmaceutical components (e.g., additional active components or inactive components such as fillers, stabilizers, solvents, or carriers) prior to administration. In certain embodiments, a pharmaceutical composition can comprise at least one additive such as a filler, bulking agent, buffer, stabilizer, or excipient. Suitable pharmaceutical additives include, e.g., mannitol, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. In certain embodiments, the pharmaceutical compositions may also contain pH buffering reagents and wetting or emulsifying agents. In some aspects, the pH buffering reagents are selected from citrate or phosphate. In some aspects the pharmaceutical compositions have a pH range of about 6.5 or higher, about 6.5 to about 8.0, about 6.5 to 7.5, or about 6.5 to 7.0. In further embodiments, the compositions may contain preservatives or stabilizers.

Also provided in various embodiments of the present invention is C1INH for use in organ infusion or allograft preparation in vitro or prior to reperfusion in a recipient, wherein the C1INH is infused into the organ or allograft within 2 hours or 60-120 minutes before the allograft is transplanted or before the organ is reperfused in the recipient.

Other embodiments provide BERINERT for use in kidney allograft preparation in vitro or prior to reperfusion in a recipient, wherein about 500 units of BERINERT in a diluent is infused into the kidney allograft on the day of transplantation at a point in time from time of induction of anesthesia up to the beginning of renal vein anastomosis before graft reperfusion in the recipient.

Further embodiments provide BERINERT for use in kidney allograft preparation in vitro or prior to reperfusion in a recipient, wherein the recipient does not need to receive a C1INH following implantation of the prepared kidney allograft. In other embodiments, a recipient of an allograft according to the present invention does not have hereditary angioedema (HAE), angioedema, or acute abdominal and/or facial angioedema.

Other embodiments provide a kit for use in organ infusion in vitro or prior to reperfusion, wherein the kit comprises a therapeutically effective amount of C1INH, a sterile diluent, and a container. In some embodiments, the C1INH is reconstituted with the sterile diluent in the kit no more than 8 hours prior to use in infusing an allograft organ, and the infused allograft organ with the reconstituted C1INH is for use within 2 hours or 60-120 minutes prior to transplantation or reperfusion in a recipient.

Further embodiments provide a kit for use in organ infusion in vitro or prior to reperfusion, wherein the kit comprise a therapeutically effective amount of C1INH, a sterile diluent, a syringe, and a needle, wherein the C1INH is reconstituted no more than 8 hours prior to use in infusing an allograft organ.

In various embodiments, instructions for use of the kits will include directions to use the kit components in the preparation of allograft prior to transplantation or prior to reperfusion with recipient's circulatory system. The instructions may further contain information regarding how to prepare (e.g. dilute or reconstitute, in the case of freeze-dried protein) the C1-Inhibitor. The instructions may further include guidance regarding the amount and frequency of infusing the allograft.

In some embodiments, a kit for use in kidney allograft infusion in vitro or prior to reperfusion comprises 500 units of BERINERT as a lyophilized concentrate in a single-use container, and a quantity of sterile diluent comprising sterile water of 5-20 mL, wherein the C1INH is reconstituted with the sterile diluent for use in infusing the kidney allograft within 2 hours or 60-120 minutes prior to transplantation or reperfusion of the kidney allograft in a recipient.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Inclusion Criteria

For purposes of the study, the following patients are eligible. (1) Adult men or women (18-70 years of age) who are on chronic dialysis therapy and acceptable candidates for receipt of a kidney transplant; (2) Recipients of kidney allograft from KDPI>80 donors; (3) Recipients of kidney allograft from DCD donors; (4) Recipients who are ABO compatible with donor allograft; (5) Understand and sign a written inform consent prior to any study specific procedure; (6) Women of childbearing potential must have a negative pregnancy test prior to randomization, and must be on an acceptable form of birth control.

For purposes of treatment outside of this study, the foregoing may not be required for patient eligibility.

### Example 2

### Exclusion Criteria

For purposes of the study, the following patients are not eligible. However, these patients are not excluded from the scope of the claims unless specifically (1) Patients with a known pro-thrombotic disorder. (e.g. Factor V Leiden); (2) Patients with a history of thrombosis or hypercoagulable state, excluding access clotting. (3) Patients with a history of administration of C1INH containing products or recombinant C1INH within 15 days prior to study entry. (4) Patients with a known hypersensitivity to treatment with C1INH. (5) Patients with an abnormal coagulation function. (INR≥2, PTT>50, PLT<60,000) who are not on anti-coagulation. (6) Patients with known active presence of malignancies. (7) Patients who are PCR positive for Hep B, Hep C, or HIV. (9) Recipients of pre-emptive kidney transplantation. (9) All zero mismatch kidneys. (10) Recipients of multi-organ transplants. (kidney and any other organ); (11) Recipients of kidney allograft that was on pump preservation for any period prior to transplantation. (12) Recipients of kidney allograft from a living donor. (13) Female subjects who are pregnant or lactating.

For purpose of treatment outside of this study, a patient with one or more of the forgoing criteria can still be eligible for treatment of the present invention.

### Example 3

### Endpoints

SAFETY: Overall incidence of adverse events and serious adverse events and relationship of AE and SAEs to the study treatment

(1) Need for HD in the 1^{st} month post-transplant; (2) eGFR at 1M, 3M, 6M & 12M; (3) Patient and graft survival at Day 365; (4) Rate of acute cellular rejection (ACR) and antibody mediated rejection at 12M.

Efficacy Assessments:
Primary Endpoints: (1) Need for Dialysis in the 1^{st} Month Post-Transplant: (Excluding patients who get dialysis for hyperkalemia) The proportion of patients enrolled who require at least one session of dialysis in the first 30 days post transplant; Number of dialysis sessions per patient in the first 30 days post transplant. (2) Renal Function and Graft Survival 12M eGFR at 12M post-transplant; Patient & Graft survival 12M post-transplant
Secondary Endpoints: Rate of acute cellular and antibody mediated rejection episodes by day 365. Calculated creatinine clearance at 1M, days 90, 180, and 365 days post transplant. Development of DSAs at 12M. SOC biopsies will be assessed at time of implantation and 12M post implantation.

### Example 4

### Immunosuppression

Induction therapy using THYMOGLOBULIN in divided doses for a total of 6 mg/kg or Campath 1H 30 mg SQ×1. Calcineurin inhibition (Tacrolimus or Cyclosporine) is delayed for minimum 24 hours post-transplant. MMF is started and used per standard of care (SOC). Corticosteroids is used per SOC. Valgancyclovir is used as the prophylaxis for CMV for a minimum of 6 months. Prophylaxis for bacterial and fungal infection is per institutional SOC. Diagnosis and treatment of acute cellular or antibody-mediated rejection is per institutional SOC.

### Example 5

### Brief Summary

This is a Phase I/II double-blind, randomized, placebo-controlled study assessing safety and limited efficacy of intraoperative C1INH (500 U/kidney) vs. Placebo administered into the graft renal artery 1-2 hours prior to implantation in adult subjects receiving a deceased donor kidney allograft considered high-risk for development of DGF (KDPI>80). Once eligible patients are identified, consented, and have an acceptable kidney transplant offer, they will be randomized by the Cedars-Sinai Research Pharmacy to receive study drug vs. placebo. Drug and placebo will be prepared by the Cedars-Sinai Research Pharmacy and conveyed to the operating room in a blinded manner. The drug will be administered by the transplant surgeon in the OR in a blinded manner.

Condition or disease: End Stage Renal Disease, Chronic Kidney Diseases; Intervention/treatment: BERINERT® (other name: C1 esterase inhibitor, C1INH), Placebo (normal saline); Phase: Phase 1, Phase 2.

### DETAILED DESCRIPTION

Pre-operative, infusion of C1INH into the renal allograft artery 1-2 hours prior to implantation will improve early graft function and reduce the rate of DGF, requirements for dialysis, graft survival and eGFR in patients receiving kidney allografts from high risk deceased donor compared to placebo.

Early graft function has a long-term effect on graft survival. Poor early graft function and DGF contributes to decreased short- and long-term patient and graft survival, increased incidence of acute rejection, prolonged hospitalization, and higher costs of transplantation. Although multiple components contribute to the impaired graft function, ischemia-reperfusion injury (IRI) is the underlying pathophysiology leading to poor early graft function and DGF. A >35% incidence of DGF has remained constant over time despite significant improvements in immunosuppressive strategies and patient management. This may be due to increased use of kidneys from “extended-criteria” and/or non-heart-beating donors, where even greater rates (>60%) of DGF have been reported.

More than 94,653 people are currently waiting for a kidney transplant in the United States (UNOS.org Sep. 30, 2019). Of the 19,360 kidney transplants performed in the US in 2018, 20% were from DCD donors and 9% from donors of KDPI>85. The USRDS reports that more than 50% of patients on the waiting list are willing to accept a kidney from an expanded-criteria donor (KDPI>85). This study will seek to expand the use of high KDPI kidneys and reduce wastage by showing improved function after C1INH treatment. Patients who fulfill all I/E criteria will be eligible to be enrolled into Study.

### Study Group (40 Patients):

Treatment Arm I—KDPI>80 kidneys will be infused with one intrarenal dose of 500 U of BERINERT® in operating room (OR) prior to implantation into the recipient.
Control Arm—KDPI>80 kidneys will be administered one intrarenal dose of normal saline (NS) in the OR in a volume identical to the volume of the dose of BERINERT® before implantation of kidney into the patient.
Drug v. placebo administration will be randomized 1:1. Drug preparation and randomization will be carried out in a blinded fashion by research pharmacist.

### Study Design

Study type: Interventional (clinical trial)
Estimated Enrollment: 40 participants
Allocation: Randomized
Intervention model: Parallel Assignment
Intervention model description: Intraoperative C1INH (500 U/kidney) vs. Placebo administered into the graft renal artery 1-2 hours prior to implantation in adult subjects receiving a deceased donor kidney allograft considered high-risk for development of DGF (KDPI>80). Patients will be randomized in a 1:1 manner
Masking: Quadruple (Participant, Care Provider, Investigator, Outcomes Assessor)
Masking description: Once eligible patients are identified, consented, and have an acceptable kidney transplant offer, they will be randomized by the Cedars-Sinai Research Pharmacy to receive study drug vs. placebo. Drug and placebo will be prepared by the Cedars-Sinai Research Pharmacy and conveyed to the operating room in a blinded manner. The drug will be administered by the transplant surgeon in the OR in a blinded manner.
Primary purpose: Prevention

### Primary Outcome Measures:

1. Need for Dialysis in the first 30 days post-transplant [Time Frame: 30 days]
   The proportion of patients enrolled who require at least one session of dialysis in the first 30 days post transplant. umber of dialysis sessions per patient in the first 30 days post transplant.
2. Renal Function 12 Months [Time Frame: 12 months]
   eGFR at 12M post-transplant.
3. Graft Survival 12 Months [Time Frame: 12 months]
   Graft survival at 12 Months.

### Secondary Outcome Measures:

1, Rejection Episodes at 12 Months [Time Frame: Month 12]
   Number of rejection episodes by day 365.
2. Renal Function at multiple time points up to 12 Months [Time Frame: Month 1, 3, 6, 12]
   Calculated creatinine clearance at 1M, days 90,180, and 365 days post transplant.
3. Development of Donor Specific Antibodies (DSA) at 12 Months [Time Frame: Month 12]
   Number of participants with Donor Specific Antibodies (DSA) at 12 Months.

### Other Outcome Measures:

1. Adverse events in the study population [Time Frame: Month 12]
   Overall incidence of adverse events and serious adverse events and relationship of AE and SAEs to the study treatment.

### Study Drug Administration Timing

In consideration of the latest time point of treatment application before graft reperfusion (i.e. beginning of renal vein anastomosis), study treatments will be administered pre-operatively at any one point in time, from time of admission up to the beginning of renal vein anastomosis before graft reperfusion. Due to the biologic half-life of C1INH (approximately 2.5 to 3.8 days), the time difference between early and late intraoperative treatment application, is thought to be negligible. This will constitute the entirety of C1INH vs. placebo administration. The study format is shown below in FIG. 1.

### Selection of Study Population

Persons legally incompetent to provide informed consent include, but may not be limited to, minors (children), individuals who are mentally incapable of understanding the implications of the PI/IC, and those who are physically unable, by any written, physical or verbal means, to confirm their understanding and consent to take part in the study. Our policy is not to enter adult patients who are mentally incompetent to give informed consent. Exceptions must be approved by the Study Director.

Up to 40 adult men and women (18-70 years of age) who are recipients of a kidney transplant and only a kidney allograft from a deceased donor who is high risk for DGF will be enrolled in the study (KDPI>80).

### Inclusion Criteria

Recipient Inclusion Criteria
1) Adult men or women (18-70 years of age) who are on chronic dialysis therapy and acceptable candidates for receipt of a kidney transplant.
2) Recipients who are ABO compatible with donor allograft.
3) Understand and sign a written informed consent prior to study specific procedures.
4) Women of childbearing potential must have a negative pregnancy test prior to randomization, and must be on an acceptable form of birth control.
5) AND one of the below criteria:
a) Recipients of kidney allograft from KDPI>80 donors
b) Recipients of kidney allograft from DCD donors
c) Recipients of kidney allograft with CIT>24 hours
d) Recipients of kidney allograft from donor on HD/CRRT prior to death/procurement
e) Recipients of kidney allograft with donor terminal creatinine SCr≥3.0 mg/dL
f) Patient risk a total risk index score of >/=3

Exclusion Criteria

While these patients are excluded from this particular study, these patient are not specifically excluded from the scope of the claims unless explicitly specified by the claims.
1) Patients with a known pro-thrombotic disorder. (e.g. Factor V Leiden)
2) Patients with a history of thrombosis or hyper-coagulable state, excluding access clotting.
3) Patients with a history of administration of C1INH containing products or recombinant C1INH within 15 days prior to study entry.
4) Patients with a known hypersensitivity to treatment with C1INH.
5) Patients with an abnormal coagulation function. (INR≥2, PTT>50, PLT<60,000) who are not on anti-coagulation.
6) Patients with known active presence of malignancies.
7) Patients who are PCR positive for Hep B, Hep C, or HIV.
8) Recipients of pre-emptive kidney transplantation.
9) All zero mismatch kidneys.
10) Recipients of multi-organ transplants. (kidney and any other organ)
11) Recipients of kidney allograft that was on pump preservation for any period prior to transplantation
12) Recipients of kidney allograft from a living donor.
13) Female subjects who are pregnant or lactating.
   Removal of Subjects from Study

A subject who is withdrawn is one who discontinued in the clinical study for any reason. Subjects may be withdrawn from the study for the following reasons: At their own request or at the request of their legally acceptable representative. If, in the investigator's opinion, continuation in the study would be detrimental to the subject's well-being. At the specific request of the sponsor. In all cases, the reason for withdrawal must be recorded in the case report form and in the subject's medical records.

Patients have the right to withdraw from the study at any time for any reason without penalty or prejudice. The Investigator also has the right to withdraw patients from the study if he/she feels it is in the best interest of the patient or if the patient is uncooperative or non-compliant. It is understood by all concerned that an excessive rate of withdrawals can render the study un-interpretable; therefore, unnecessary withdrawal of patients should be avoided. Should a patient decide to withdraw, all efforts will be made to complete and report the observations, and early withdrawal procedures, as thoroughly as possible.

The Investigator should contact the patient either by telephone or through a personal visit to determine as completely as possible the reason for the withdrawal. A complete final evaluation at the time of the patient's withdrawal should be made with an explanation of why the patient is withdrawing from the study. If the reason for removal of a patient from the study is an adverse event or an abnormal laboratory test result, the principal specific event or test will be recorded on the CRF. For all patients who are withdrawn pre-maturely, every attempt should be made to assess the patient's status (i.e. patient and graft survival, malignancies, etc.) at 30 days after administration of the last dose of study medication.

### Treatments to be Administered

This study is designed with two blinded treatment groups (C1INH vs. Placebo). Each group will include 20 patients in a treatment arm and 20 patients in a placebo arm.

This design is statistically powered to show significant differences if there is a 30-50% reduction in the treatment group and may trend towards positive treatment effect since extensive DGF (40-50%) is expected in KDPI>80 recipients. The dose of C1INH chosen is (500 U/kidney) (high-dose treatment) which should be sufficient to bind all endothelial surfaces in the transplanted kidney. In those randomized to placebo, an equivalent volume of placebo (0.9% normal saline) will be administered prior to graft reperfusion. Both groups (C1INH or placebo) will receive an intrarenal administration of heparinized normal saline before study drug administration.

Patients who fulfill all FE criteria will be eligible to be enrolled into this Phase I/II study.

### Identity of Investigational Product(s)

Medication will be labeled according to the requirements of local law and legislation. Label text will be approved according to agreed CSMC Research Pharmacy procedures.

Product name: BERINERT®/C1INH (Human); Chemical name: Complement Component 1 Inhibitor (C1INH); Study Medication & Dosing: BERINERT® is available in a single-use vial that contains 500 units of C1 esterase inhibitor as a lyophilized concentrate. Each vial must be reconstituted with 10 mL of diluent (sterile water) provided. Prior to reconstitution, BERINERT® should be stored at 2° to 25° C. After reconstitution, administration may begin within 8 hours provided the solution has been stored at up to 25° C. BERINERT® is dosed as a 500 U/kidney infusion 1-2 hours prior to implantation of the KDPI>80% kidney. Participating patients will receive 500 U/kidney C1INH vs placebo (0.9% NS) prior to implantation. The C1INH or placebo dose will be preceded by an intrarenal administration of heparinized normal saline.

### Method of Assigning Subjects to Treatment Groups

Patients will be randomized into treatment/placebo arms in a 1:1 ratio. Randomization will be completed prior to patient entering the operating room for transplantation.

This study is a double-blind, placebo-controlled study. The clinical pharmacist will maintain the randomization assignment. The Investigators, other study personnel, patients, and the Sponsor will be blinded to treatment assignment.

### BERINERT® (C1INH Dosing)

For the planned study, subjects will receive C1NH 500 U/kidney infused into the renal artery pre-operatively at any one point in time, from time of induction of anesthesia up to the beginning of renal vein anastomosis before graft reperfusion. For all dose reconstitution, BERINERT® is provided in single dose vials of 500 units. Each vial will be reconstituted in 10 ml sterile water for injection. Placebo will be administered in an identical volume. C1INH will be preceded by an intrarenal administration of heparinized normal saline prior to renal vein anastomosis.

### Placebo Dosing

The placebo used in this study will be 0.9% normal saline administered as a single into the renal artery infusion at any one point in time, from time of induction of anesthesia up to the beginning of renal vein anastomosis before graft reperfusion. The total volume will be identical to that calculated for BERINERT® infusions. Placebo will be preceded by an intrarenal administration of heparinized normal saline prior to renal vein anastomosis.

### Immunosuppression

Immunosuppression regimen is an important part of the care for recipients of kidney transplantation. The regimen to be used in this study is reflecting the fact that the patients enrolled into this study are at high risk for developing poor or delayed graft function.

### Induction Antibody Therapy

All patients enrolled into this study will be treated with induction antibody therapy with THYMOGLOBULIN® or Campath 1H. For THYMOGLOBULIN®, the first dose will be given immediately after the transplantation procedure to follow with additional doses so that patients receive up to 6 mg/kg (1.5 mg/kg/dose) using standard institutional treatment guidelines for use of this agent in this patient population. Campath 1H will be administered in a single 30 mg subcutaneous dose post-transplant. No induction administration of IL-2 receptor inhibitors is permitted in this protocol.

### Maintenance Immunosuppression

Calcineurin Inhibitors (Tacrolimus or Cyclosporine). Tacrolimus (Prograf) and Cyclosporine (Neoral) are indicated for the prophylaxis of organ rejection in patients receiving allogeneic kidney transplants. Tacrolimus or Cyclosporine will be initiated and monitored with trough levels using standard institutional treatment guidelines for use of these agents in this patient population.

Mycophenolate Mofetil (MMF). Mycophenolate mofetil (MMF, CellCept), the morpholinoethylester of mycophenolic acid, has been shown to have antiproliferative effects on lymphocytes by blocking proliferation of T- and B-lymphocytes. MMF will be dosed using standard institutional treatment guidelines for use of these agents in this patient population. Myfortic can be used instead of CellCept and will be dosed using standard institutional treatment guidelines for use of these agents in this patient population.

Corticosteroids. Corticosteroids will be administered using standard institutional treatment guidelines for use of these agents in this patient population.

### Anti-Infective Prophylaxis

Valgancyclovir for the prophylaxis of CMV infection will be used for a minimum of 6 months. If PO administration of valgancyclovir is not possible, the use of ganciclovir while the patient is inpatient is also permissible.

Penicillin VK 500 mg po bid will be administered ×7 days (up to 7 days while inpatient or until patient is discharged from the facility) for meningococcal prophylaxis (or alternate prophylactic antibiotic for penicillin allergic patients)

Local standards of care for post-transplant bacterial and fungal prophylaxis will be applied using standard institutional treatment guidelines for use of these agents in this patient population.

### Selection and Timing of Dose for Each Subject

The active treatment arm (BERINERT® 500 U/kidney) will be compared to a placebo control treatment arm, with a total of 20 adult subjects in each study group. A total of 40 adult subjects will be enrolled in the study and additional subjects will not be enrolled to replace dropouts. This design is statistically powered to show differences in DGF with a 30-50% reduction in DGF predicted for the BERINERT® study group. Treatment effect will be investigated following renal artery infusion with BERINERT® or an equivalent volume of placebo (0.9% normal saline) pre-operatively prior to graft reperfusion. The C1INH or placebo dose will be preceded by an intrarenal administration of heparinized normal saline.

### Blinding

The pharmacist will be contacted by the study staff and informed of a patient's eligibility. The pharmacist will prepare the blinded study medication or placebo. The study treatment will be prepared according to a pharmacist-supplied randomization code (blinded from all other study personnel). The study treatment will be prepared in a non-siliconized sterile syringe (BERINERT® {500 U} for all individuals) and labeled with: Protocol Number, Screening number, Randomization number.

The pharmacist will check the non-siliconized IV syringe for potential particulates. If none are found, the blinded syringe of study treatment, labeled as stated above, will be transported to the surgical suite and matched with the matching patient chart. For the placebo arm, an equivalent volume of normal saline will be prepared in an identical dispensing medium as the C1INH product, labeled as stated above, and transported to the surgical suite and matched with the matching patient chart. Before the study treatment (BERINERT or placebo), heparinized normal saline will be administered to the allograft. This method of drug administration has not been FDA approved and may have unforeseen risks

In case of a medical emergency, the study drug assignment for a particular patient may be unblinded. Unblinding study drug assignment will only be necessary if knowledge about treatment is needed for the medical management of the patient. The patient's treatment assignment can be identified for unblinding purposes by calling the Investigational Pharmacist (310-423-6580). When this is necessary, the investigator must immediately notify the IRB, and document the reason and date of the un-blinding. The event must also be documented on the study termination record, the AE page of the CRF, and in source documents. Additionally, the Principal Investigator will submit to the IRB a written explanation describing the event within 5 working days. The procedures for un-blinding study drug treatment will be provided to the Principal Investigators prior to study implementation.

### Treatment Compliance

There is only one application of the drug, which takes place approximately 60-120 minutes prior to graft reperfusion administered pre-operatively.

### Primary Efficacy Endpoints

For patients who require dialysis in the first 30 days: (Excluding patients who get dialysis for hyperkalemia): The proportion of patients enrolled who require at least one session of dialysis in the first 30 days post transplant; Number of dialysis sessions per patient in the first 30 days post-transplant.

Renal function and graft survival 12M. eGFR at 12M post-transplant. Patient and graft survival at 12M post-transplant

### Secondary Efficacy Endpoints

Calculated Creatinine Clearance obtained at 1M, 3M, 6M and 12M post-transplant. Rate of acute cellular and antibody mediated rejection episodes by day 365. Development of DSAs at 12M. Analysis of renal transplant biopsies at time of implantation and at 12M as clinically indicated.

### Safety Variables

Adverse Drug Reactions: The most serious adverse reaction reported in subjects in clinical studies who received BERINERT® was an increase in the severity of pain associated with hereditary angioedema. In a placebo controlled clinical study, the incidence of adverse events occurring in more than 4% of subjects (n=43) receiving BERINERT® up to 72 hours after infusion was nausea (7%), headache (7%), abdominal pain (7%), dysgeusia (4.7%), vomiting (2.3%), pain (2.3%), and muscle spasms (2.3%). Adverse reactions can persist 7-9 days after infusion and have been reported to be abdominal pain (6.5%), diarrhea (4.6%), nausea (6.5%), vomiting (4.6%), headache (11.1%), hereditary angioedema recurrence (11.1%), muscle spasm (5.6%), and pain (5.6%). Subjects were tested at baseline and after 3 months for exposure to parvovirus B19, HBV, HCV, and HIV-1 and HIV-2. No subject who underwent testing evidenced seroconversion or treatment-emergent positive polymerase chain reaction testing for the above pathogens. Post-marketing reports from Europe since 1979 in patients receiving BERINERT® for treatment of HAE include hypersensitivity/anaphylactic reactions, a few suspected cases of viral transmission, including cases of acute hepatitis C, injection-site pain, injection-site redness, chills, and fever. See Novation Drug Monograph for BERINERT®. BERINERT® was well tolerated with minimal AE/SAE profile in our kidney transplant studies.

Assessment of Risk for Thrombotic Events with C1INH Administration: Patients with known risk factors for thrombotic events will be monitored for signs and symptoms of thrombosis, such as new onset swelling and pain in the limbs or abdomen, new onset chest pain, shortness of breath, loss of sensation or motor power, or altered consciousness, vision, or speech. Patients will be assessed with the infusion for evidence of DVT or other TEs. In addition, routine measurements of coagulation factors to evaluate DIC (D-Dimers, fibrinogen, PT, PTT) will be performed.

Evidence of a Thrombotic Effect of C1INH Administration when Given in Supraphysiologic Dosages to Humans: The German Medical Profession's Drugs Committee (AkdA) reported on 13 cases of severe thrombus formation after BERINERT® infusions. These infusions were given to neonates undergoing cardiac surgery and doses of BERINERT® were given at exceedingly high doses (˜500 U/kg) without control studies. Nine of these infants died of this complication. The conclusions from this report are as follows:

There are no studies to support the use of C1INH outside of those with hereditary angioneurotic edema. The use of C1INH in patients who do not have C1INH deficiency may result in abnormal coagulation parameters with propensity to thrombosis. There are no controlled studies that suggests a benefit of C1INH therapy in conditions other than C1INH deficiency. Any proposed uses outside of C1INH deficiency should be conducted in a controlled manner to monitor for unexpected or unanticipated side effects of C1INH therapy.

This is a serious and unanticipated complication of C1INH therapy that could potentially limit its use in patients with other conditions where complement inhibition would be desirable. This is the case with our proposal. Inhibition of complement activation post-transplant has great potential for prevention of complement-dependent DGF. In addition, there is an important unmet need for new therapeutics in the prevention and treatment of DGF. Despite this, it is important to assess the tolerability and safety of C1INH therapy in this patient population. Although the C1INH dosing proposed in our study is much less than that reported to be associated with TE and well below therapeutic dosing recommendations (20-50 U/kg) (500 U/kidney once on Day 0), there is still a possibility that increasing C1INH levels above normal baseline could have deleterious effects and induce coagulation abnormalities. Recent safety data from clinical trials of C1INH for use in C1INH deficiency have not reported TE with one exception of a basilar artery thrombosis in a patient treated for C1INH deficiency. This event was deemed unrelated to C1INH therapy. As previously indicated if significant coagulation abnormalities or TE events are seen in any study patient, the study will be terminated.

Evidence of a Thrombotic Effect of C1INH Administration when Given in Supra-physiologic Dosages to Animals: Data on C1INH transgenic mice show that blood levels as high as 2 mg/ml (NL 25 μg/ml) are produced without reported effects. Other investigators have shown that ischemia-reperfusion injury is reduced in animals transgenic for C1INH expression. Data provided by CSL Behring shows that administration of BERINERT® up to 200 U/kg daily for 14 days had no deleterious effects and did not induce coagulation abnormalities.

Therapy Stopping Points. As indicated previously, the study will be halted and re-evaluated by the DSMB if any patient in the study group develops thrombosis of the allograft, thromboembolic events (TE) or evidence of coagulation abnormalities that would suggest impending TE. In addition, the study will be halted or stopped if any other known or unexpected AEs attributable to C1-inhibitor occur. Reassessment of the study goals and complications will be done and discussed with the DSMB prior to proceeding. A summary report will be submitted to the IRB and FDA.

Primary Safety Endpoints. Overall incidence of adverse events and serious adverse events and relationship to the study treatment including but not limited to, occurrence of infections, thrombosis of the allograft, vascular thromboses, bleeding, death, vital signs, and abnormal lab values.

Secondary Safety Endpoints. Chemistry and coagulation parameters. eGFR at 1M, 3M, 6M, 12M. Patient survival at day 365. Graft survival at day 365. Rate of acute cellular and antibody mediated rejection episodes at day 365. Rate of de novo Donor Specific Antibody (DSA) development

### Assessment Periods

Screening Procedure—Day 0. Prior to screening activities, each patient must be given an opportunity to ask questions and to understand the details of study participation. This consent process must be documented in the patient's source documents and evidenced by the patient signing the informed consent form.

After signing the ICF, each patient will be assigned a patient identifier number that will be used on all subject documentation. Numbers will be assigned in ascending sequential order. This number will also correspond to the patient number entered on study materials.

The Principal Investigator or qualified and assigned Sub-investigator will review the inclusion and exclusion criteria and laboratory test data to confirm eligibility of each subject.

The screening procedures will include the following: Informed consent; Medical history; Inclusion/Exclusion criteria review; Vital signs/weight; Complete physical examination; Hematology & chemistry profile; PT, PTT, D-Dimer, Fibrinogen; Review historical serologies for HIV, HBV, HCV, CMV and EBV; Pregnancy test (for WOCP); Urine output measurement; Urinalysis; Concomitant medications; Pneumococcal vaccine

Day 0—Day of Transplantation. Inclusion/Exclusion criteria (pre-transplant); Vital signs (pre- and post-transplant); Physical examination (pre- and post-transplant); Randomization (pre-transplant); Hematology & chemistry profile (pre- and post-transplant); PT, PTT and INR (pre- and post-transplant); Concomitant medications (pre- and post-transplant); ECG (pre-transplant); Chest x-ray (pre-transplant); Serum Creatinine (pre- and post-transplant, within 4-hours of surgery); Clinical Assessment (post-transplant); BERINERT vs. Placebo infusion (on call to OR); C1 inhibitor, C3, C4 levels (prior to dose #1 of C1INH or placebo); Campath 1H or THYMOGLOBULIN administration; Assessment of implantation biopsy (if collected)

Day 1-7 Assessments measured daily through day 7 (or until discharge). Concomitant medications; Serum creatinine measurements daily through Day 7 or until discharge; Dialysis assessment; Vital Signs; Clinical Assessment; Calculated Creatinine/Calculated GFR; Urine output measurement; Adverse event assessment; Hematology & chemistry profile

Day 30±5 days. Concomitant medications; Serum creatinine measurement; Vital Signs; Dialysis assessment; Adverse Event Assessment; Clinical Assessment; Calculated Creatinine/Calculated GFR; Hematology & chemistry profile

Days 90, 180 and 365±15 days. Concomitant medications; Serum creatinine measurement; Dialysis assessment; Vital Signs; Adverse Event Assessment; Clinical Assessment; Calculated Creatinine/Calculated GFR; Hematology & chemistry profile; ACR assessment on Day 365; Assessment of SOC biopsies on Day 365; Patient survival assessment on day 365; Graft survival assessment on day 365; Donor Specific Antibody assessment at Day 365

### Physical Examination

A complete physical examination will include; body weight and the examination of the following body systems: general appearance, skin, HEENT (head, ears, eyes, nose, throat), cardiovascular, pulmonary, abdomen, neurological, lymph nodes, spine and extremities (skeletal).

### Clinical Assessment

A clinical assessment will be performed and will include a limited physical examination, the evaluation of the patient for clinical signs of infection, possible rejection, adverse events, and change in renal function. Any abnormalities will be recorded on the CRF.

### Vital Signs

Vital signs; including blood pressure, heart and respiratory rate will be measured using clinically acceptable methods and devices at each clinic visit. Height will be measured at the screening visit only.

### Serum Creatinine

Serum creatinine levels will be measured per institutional SOC.

### Calculated Creatinine Clearance/Calculated GFR

Calculated creatinine clearance and GFR will be calculated using the MDRD or Cockroft & Gault formula.

### Urine Output

Urine output will be collected and recorded for the first 5-7 days post-transplant.

### PT, PTT and INR Tests

PT, PTT and INR tests will be performed per institutional SOC.

### Hematology and Chemistry Profile

Hematology and Chemistry profile performed per institutional SOC and will include measurements of WBC, RBC, platelets, electrolytes, BUN, glucose, T Bili, Alb, Alk Phos, AST, ALT, BUN and Albumin.

### ECG Testing

An ECG will be performed per institutional SOC.

### ACR Assessment

An acute rejection assessment will be recorded on patients who were diagnosed with an ACR grade ≥Grade 2 per the Banff grading system and received anti-rejection therapy.

### Rate and Duration of Dialysis

Duration of dialysis will be measured by the number of sessions of dialysis treatment needed in the first 30 days post transplant. As for rate of dialysis, one dialysis session will represent the need for dialysis treatment.

### Data Quality

Monitoring and auditing procedures defined/agreed by the sponsor will be followed, in order to comply with Good Clinical Practice (GCP) guidelines. Our center will be monitored at quarterly to ensure compliance with the study protocol, GCP and legal aspects. This will include on-site checking of the case report forms (CRF) for completeness and clarity, cross checking with source documents, and clarification of administrative matters.

### Documentation

Entries made in the CRF must be either verifiable against source documents, or have been directly entered into the CRF, in which case the entry in the CRF will be considered as the source data. The source data parameter to be verified and the identification of the source document must be documented. The study file and all source data should be retained until notification given by the sponsor for destruction.

### Adverse Events (AEs)

An AE is any untoward medical occurrence in a subject or clinical investigation subject administered with a pharmaceutical product. The AE does not necessarily have to have a causal relationship with this treatment. An AE can therefore be any unfavorable and unintended sign (including an abnormal laboratory finding), symptom, or disease temporally associated with the use of an investigational product, whether or not considered related to the medicinal product.

Adverse events associated with the use of a drug in humans, whether or not considered drug related, include the following:

An AE occurring in the course of the use of a drug product in professional practice, An AE occurring from an overdose whether accidental or intentional, An AE occurring from drug abuse, An AE occurring from drug withdrawal. An AE where there is a reasonable possibility that the event occurred purely as a result of the subject participation in the study (e.g. adverse event or serious adverse event due to discontinuation of anti-hypertensive drugs during wash-out phase) must also be reported as an adverse event even if it is not related to the investigational product.

The clinical manifestation of any failure of expected pharmacological action is not recorded as an AE if it is already reflected as a data point captured in the CRF. If, however, the event fulfills any of the criteria for a “serious” AE (SAE), it must be recorded and reported as such.

### Serious Adverse Event (SAE)

An SAE is any untoward medical occurrence that at any dose: results in death, is life-threatening, requires in-patient hospitalization or prolongation of existing hospitalization, results in persistent or significant disability or incapacity, is a congenital anomaly or birth defect, is an important medical event

Life-threatening: The term “life-threatening” in the definition of “serious” refers to an adverse event in which the subject was at risk of death at the time of the event. It does not refer to an adverse event which hypothetically might have caused death if it were more severe.

Hospitalization: Any AE leading to hospitalization or prolongation of hospitalization will be considered as Serious, UNLESS at least one of the following exceptions are met: The admission is pre-planned (i.e., elective or scheduled surgery arranged prior to the start of the study); OR The admission is not associated with an adverse event (e.g., social hospitalization for purposes of respite care)

However, it should be noted that invasive treatment during any hospitalization may fulfill the criterion of ‘medically important’ and as such may be reportable as an SAE dependent on clinical judgment. In addition, where local regulatory authorities specifically require a more stringent definition, the local regulation takes precedent.

Disability: a substantial disruption of a person's ability to conduct normal life's functions.

Important medical event: Any adverse event may be considered serious because it may jeopardize the subject and may require intervention to prevent another serious condition. As guidance for determination of important medical events refer to the “WHO Adverse Reaction Terminology—Critical Terms List”. These terms either refer to or might be indicative of a serious disease state.

Such reported events warrant special attention because of their possible association with a serious disease state and may lead to more decisive action than reports on other terms.

### Unexpected Adverse Event (AE)

An unexpected AE is any adverse drug event, the specificity or severity of which is not consistent with the current Investigator Brochure (or Package Insert for marketed products). Also, reports which add significant information on specificity or severity of a known, already documented adverse event constitute unexpected AEs. For example, an event more specific or more severe than described in the Investigator Brochure would be considered “unexpected”. Specific examples would be; (a) acute renal failure as a labeled adverse event with a subsequent new report of interstitial nephritis and (b) hepatitis with a first report of fulminant hepatitis.

Calculation of GFR from age, gender, race, urea, creatinine and albumin (MDRD equation) (Levey et al, 1999; Tattersall, 2003).
Albumin in g/dL, age in years. GFR in mL/min/1.73 m².
Multiply by 1.18 if patient is black. Multiply by 0.762 if patient is female.
SI units (Creatinine in μmol/L, Urea in mmol/L)

GFR=170×(Creat×0.0113)^{-0.999}×age^{-0.176}×(Urea×2.8)^{-0.17}×Alb^{-0.318}
US units (Creatinine in mg/dL, BUN in mg/dL)

GFR=170×Creat^{-0.999}×age^{-0.176}×BUN^{-0.17}×Alb^{-0.318}

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A method of reducing delayed graft function, ischemia/reperfusion injury, or improving long-term graft function, comprising:
administering a therapeutically effective dose of complement component 1 esterase inhibitor (C1INH) to an allograft prior to implantation of the allograft into a subject or prior to allograft reperfusion in the subject in need thereof, or both.

2. The method of claim 1, further comprising administering saline to the allograft prior to administering the therapeutically effective dose of C1INH.

3. The method of claim 1, further comprising one or more of implanting the allograft into a subject in need thereof, administering an immunosuppression therapy to the subject, and administering an anti-infective to the subject, and wherein the immunosuppression therapy comprises an induction antibody therapy, calcineurin inhibitor, mycophenolate mofetil (MMF), and/or corticosteroid, and optionally wherein the anti-infective comprises valgancyclovir, ganciclovir, or penicillin.

4-7. (canceled)

8. The method of claim 1, further comprising administering C1INH to the subject at the time of transplantation, at about 1 day post transplantation, or both.

9. The method of claim 1, wherein administering the therapeutically effective dose of C1INH to the allograft comprises infusing into the renal artery.

10. The method of claim 1, wherein the C1INH is plasma-derived complement component 1 inhibitor, or BERINERT®.

11. The method of claim 1, wherein the therapeutically effective dose of C1INH to the allograft is about 250-1000 U/allograft.

12. The method of claim 11, wherein the therapeutically effective dose of C1INH is about 500 U/allograft.

13. The method of claim 1, wherein the therapeutically effective dose of C1INH to the allograft is administered about 1-2 hours prior to implantation of the allograft.

14. (canceled)

15. The method of claim 3, wherein the method comprises the administration of the induction antibody therapy, and wherein the induction antibody therapy is an anti-thymocyte globulin and the first dose of the anti-thymocyte globulin is administered immediately after the transplantation of the allograft followed with additional doses of the anti-thymocyte globulin, optionally the total dose of the anti-thymocyte globulin being about 6 mg/kg of the subject.

16. (canceled)

17. The method of claim 3, wherein the method comprises the administration of the induction antibody therapy, and wherein the induction antibody therapy is Campath 1H and is administered in a single 30 mg subcutaneous dose post-transplant.

18. (canceled)

19. The method of claim 1, wherein the allograft is a kidney.

20. The method of claim 19, wherein the kidney is a kidney allograft having a kidney donor profile index (KDPI) of 80% or greater.

21. The method of claim 20, wherein the KDPI is 85% or greater.

22. The method of claim 21, wherein the KDPI is 90% or greater.

23. The method of claim 19, wherein the kidney is a kidney allograft from a donor after cardiac death.

24. The method of claim 19, wherein the kidney is a kidney allograft with a cold ischemia time (CIT) of 24 hours or longer.

25. The method of claim 19, wherein the kidney is a kidney allograft from a donor on hemodialysis or renal replacement therapy prior to death of the donor or procurement of the kidney.

26. The method of claim 19, wherein the kidney is a kidney allograft with donor terminal serum creatinine level of 3.0 mg/dL or greater.

27. The method of claim 19, wherein the C1 INH is administered to the allograft after the allograft is grafted but before reperfusion in the subject in need thereof.

28. (canceled)
